SEQUENCE LISTING (96 sequences)

SEQ ID NO: 1 - protein, 30 aa, artificial sequence
  FR1
DVQLQESGGGSVQAGGSLRLSCAASGVAHR

SEQ ID NO: 2 - protein, 14 aa, artificial sequence
  FR2
WFRQAPGKEREGVA

SEQ ID NO: 3 - protein, 32 aa, artificial sequence
  FR3
RFTISQDNANDMVYLQMLSLKPEDTAMYYCAA

SEQ ID NO: 4 - protein, 11 aa, artificial sequence
  FR4
WGQGTQVTVSS

SEQ ID NO: 5 - protein, 5 aa, artificial sequence
  CDR1
RYYMS

SEQ ID NO: 6 - protein, 17 aa, artificial sequence
  CDR2
SIYIGSGSTDYVDSVKG

SEQ ID NO: 7 - protein, 14 aa, artificial sequence
  CDR3
GLYGGRWDRAQYKY

SEQ ID NO: 8 - protein, 30 aa, artificial sequence
  FR1
DVQLQESGGGSVQAGGSLRLSCTASGYTRS

SEQ ID NO: 9 - protein, 15 aa, artificial sequence
  FR2
WFRQAPGKEREGVAA

SEQ ID NO: 10 - protein, 35 aa, artificial sequence
  FR3
RFTISQDNAKNAKNTVYLQMNSLKPEDTAMYYCAA

SEQ ID NO: 11 - protein, 11 aa, artificial sequence
  FR4
WGQGTQVTVSS

SEQ ID NO: 12 - protein, 5 aa, artificial sequence
  CDR1
TIWIG

SEQ ID NO: 13 - protein, 16 aa, artificial sequence
  CDR2
IYIGSGATYYVDSVKG

SEQ ID NO: 14 - protein, 19 aa, artificial sequence
  CDR3
TGGTVGSMVRFSPVGDFGY

SEQ ID NO: 15 - protein, 30 aa, artificial sequence
  FR1
DVQLQESGGGLVQPGGSLRLSCAASGFTFT

SEQ ID NO: 16 - protein, 14 aa, artificial sequence
  FR2
WVRQAPGKGLEWVS

SEQ ID NO: 17 - protein, 32 aa, artificial sequence
  FR3
RSTISRDNAKNTLYLQLNSLETEDTAMYYCAK

SEQ ID NO: 18 - protein, 11 aa, artificial sequence
  FR4
RGQGTQVTVSS

SEQ ID NO: 19 - protein, 5 aa, artificial sequence
  CDR1
NYAMS

SEQ ID NO: 20 - protein, 16 aa, artificial sequence
  CDR2
GINSDGGTYYSNSVKG

SEQ ID NO: 21 - protein, 15 aa, artificial sequence
  CDR3
GNGPSWTLGSVDVNS

SEQ ID NO: 22 - protein, 30 aa, artificial sequence
  FR1
DVQLQESGGGSVQAGGSLRLSCAASGDTYR

SEQ ID NO: 23 - protein, 32 aa, artificial sequence
  FR3
RFTISQDSAKNTVYLQMNSLKPEDTAIYYCAA

SEQ ID NO: 24 - protein, 5 aa, artificial sequence
  CDR1
RNTMG

SEQ ID NO: 25 - protein, 17 aa, artificial sequence
  CDR2
AIYTGGTSTAYADSVRG

SEQ ID NO: 26 - protein, 17 aa, artificial sequence
  CDR3
GSVWSRYTWSWPSTYSY

SEQ ID NO: 27 - protein, 30 aa, artificial sequence
  FR1
DVQLQESGGGSVQAGGSLRLSCAASEYSYS

SEQ ID NO: 28 - protein, 32 aa, artificial sequence
  FR3
RFTISQDIAENTVYLRMNSLKPEDTAMYYCAA

SEQ ID NO: 29 - protein, 5 aa, artificial sequence
  CDR1
RKWMG

SEQ ID NO: 30 - protein, 16 aa, artificial sequence
  CDR2
AIYTSGRTAVADSVKG

SEQ ID NO: 31 - protein, 16 aa, artificial sequence
  CDR3
LTGASDSPLRPSEYNY

SEQ ID NO: 32 - protein, 30 aa, artificial sequence
  FR1
DVQLQESGGGSVQAGGSLRLSCAASGFTFS

SEQ ID NO: 33 - protein, 32 aa, artificial sequence
  FR3
RFTISRDNAKNTMYLQMNSLKPEDTAMYYCAA

SEQ ID NO: 34 - protein, 5 aa, artificial sequence
  CDR1
SYDMS

SEQ ID NO: 35 - protein, 17 aa, artificial sequence
  CDR2
AINSGGGSTYYADSVKG

SEQ ID NO: 36 - protein, 21 aa, artificial sequence
  CDR3
DPGSSLAVGAWLKAADPFYGY

SEQ ID NO: 37 - protein, 19 aa, artificial sequence
  CDR3
TSGTVGSMVRFSPVGDFGY

SEQ ID NO: 38 - protein, 30 aa, artificial sequence
  FR1
DVQLQESGGGLVQPGGSLRLSCAASGFTFS

SEQ ID NO: 39 - protein, 14 aa, artificial sequence
  FR2
WVRQAPGRGLEWVS

SEQ ID NO: 40 - protein, 32 aa, artificial sequence
  FR3
RFTISRDNAKKMLYLQMDSLKPEDTAVYYCAT

SEQ ID NO: 41 - protein, 5 aa, artificial sequence
  CDR1
GVDMS

SEQ ID NO: 42 - protein, 17 aa, artificial sequence
  CDR2
GFGSGGESPSYADSVKG

SEQ ID NO: 43 - protein, 6 aa, artificial sequence
  CDR3
RWAEGY

SEQ ID NO: 44 - protein, 30 aa, artificial sequence
  FR1
DVQLQESGGGLVQPGGSLRLSCAASGFLFS

SEQ ID NO: 45 - protein, 32 aa, artificial sequence
  FR3
RFTISRDNAKNTLTLQLNSLKTEDTAMYYCAK

SEQ ID NO: 46 - protein, 5 aa, artificial sequence
  CDR1
RYSMS

SEQ ID NO: 47 - protein, 17 aa, artificial sequence
  CDR2
GITNGGSITKYADSVKG

SEQ ID NO: 48 - protein, 15 aa, artificial sequence
  CDR3
GAGEYGPVSDYVFSL

SEQ ID NO: 49 - protein, 30 aa, artificial sequence
  FR1
DVQLQESGGGSVQAGGSLRLSCAASGYTIG

SEQ ID NO: 50 - protein, 32 aa, artificial sequence
  FR3
RFAISEDNAKNTVYLHMNLLKPEDTAMYYCAA

SEQ ID NO: 51 - protein, 5 aa, artificial sequence
  CDR1
SNYMG

SEQ ID NO: 52 - protein, 17 aa, artificial sequence
  CDR2
AIYTGGGNTYYADSVKG

SEQ ID NO: 53 - protein, 16 aa, artificial sequence
  CDR3
DLRLRVAVLHPYGYNY

SEQ ID NO: 54 - protein, 30 aa, artificial sequence
  FR1
DVQLQESGGGLVQPGGSLRLSCAVSGFTFS

SEQ ID NO: 55 - protein, 15 aa, artificial sequence
  FR2
SWVRQAPGKGLEWVS

SEQ ID NO: 56 - protein, 32 aa, artificial sequence
  FR3
RFTISRDNAKNTLYLQLNNLKTEDTAMYYCTK

SEQ ID NO: 57 - protein, 11 aa, artificial sequence
  FR4
GGQGTQVTVSS

SEQ ID NO: 58 - protein, 4 aa, artificial sequence
  CDR1
NYAM

SEQ ID NO: 59 - protein, 16 aa, artificial sequence
  CDR2
RISSSGITYYADSVKG

SEQ ID NO: 60 - protein, 5 aa, artificial sequence
  CDR3
SSEAP

SEQ ID NO: 61 - protein, 30 aa, artificial sequence
  FR1
DVQLQESGGGSVQAGGSLRLSCAASGAIYR

SEQ ID NO: 62 - protein, 32 aa, artificial sequence
  FR3
RFTISQDNAKNTVYLQMNDLKPEDTAMYYCAA

SEQ ID NO: 63 - protein, 5 aa, artificial sequence
  CDR1
GNYMG

SEQ ID NO: 64 - protein, 17 aa, artificial sequence
  CDR2
AIATNGRTTYYADSVKG

SEQ ID NO: 65 - protein, 19 aa, artificial sequence
  CDR3
DNSWSCTVVGGLLAPAYNY

SEQ ID NO: 66 - protein, 30 aa, artificial sequence
  FR1
DVQLQESGGGSVQAGGSLRLSCAASRFTYS

SEQ ID NO: 67 - protein, 32 aa, artificial sequence
  FR3
RFTISLDIAKKTLSLQMDSLKSEDTAMYYCAA

SEQ ID NO: 68 - protein, 5 aa, artificial sequence
  CDR1
WYCMG

SEQ ID NO: 69 - protein, 17 aa, artificial sequence
  CDR2
AICTTPSRTYAADSVKG

SEQ ID NO: 70 - protein, 18 aa, artificial sequence
  CDR3
DTRGAIGFFKGSGPAIPY

SEQ ID NO: 71 - protein, 123 aa, artificial sequence
  1# nanobody
DVQLQESGGGSVQAGGSLRLSCAASGVAHRRYYMSWFRQAPGKEREGVASIYIGSGSTDYVDSVKGRFTISQDNANDMVYLQMLSLKPEDTAMYYCAAGLYGGRWDRAQYKYWGQGTQVTVSS

SEQ ID NO: 72 - protein, 131 aa, artificial sequence
  2# nanobody
DVQLQESGGGSVQAGGSLRLSCTASGYTRSTIWIGWFRQAPGKEREGVAAIYIGSGATYYVDSVKGRFTISQDNAKNAKNTVYLQMNSLKPEDTAMYYCAATGGTVGSMVRFSPVGDFGYWGQGTQVTVSS

SEQ ID NO: 73 - protein, 123 aa, artificial sequence
  3# nanobody
DVQLQESGGGLVQPGGSLRLSCAASGFTFTNYAMSWVRQAPGKGLEWVSGINSDGGTYYSNSVKGRSTISRDNAKNTLYLQLNSLETEDTAMYYCAKGNGPSWTLGSVDVNSRGQGTQVTVSS

SEQ ID NO: 74 - protein, 126 aa, artificial sequence
  4# nanobody
DVQLQESGGGSVQAGGSLRLSCAASGDTYRRNTMGWFRQAPGKEREGVAAIYTGGTSTAYADSVRGRFTISQDSAKNTVYLQMNSLKPEDTAIYYCAAGSVWSRYTWSWPSTYSYWGQGTQVTVSS

SEQ ID NO: 75 - protein, 124 aa, artificial sequence
  5# nanobody
DVQLQESGGGSVQAGGSLRLSCAASEYSYSRKWMGWFRQAPGKEREGVAAIYTSGRTAVADSVKGRFTISQDIAENTVYLRMNSLKPEDTAMYYCAALTGASDSPLRPSEYNYWGQGTQVTVSS

SEQ ID NO: 76 - protein, 130 aa, artificial sequence
  6# nanobody
DVQLQESGGGSVQAGGSLRLSCAASGFTFSSYDMSWVRQAPGKGLEWVSAINSGGGSTYYADSVKGRFTISRDNAKNTMYLQMNSLKPEDTAMYYCAADPGSSLAVGAWLKAADPFYGYWGQGTQVTVSS

SEQ ID NO: 77 - protein, 131 aa, artificial sequence
  7# nanobody
DVQLQESGGGSVQAGGSLRLSCTASGYTRSTIWIGWFRQAPGKEREGVAAIYIGSGATYYVDSVKGRFTISQDNAKNAKNTVYLQMNSLKPEDTAMYYCAATSGTVGSMVRFSPVGDFGYWGQGTQVTVSS

SEQ ID NO: 78 - protein, 115 aa, artificial sequence
  8# nanobody
DVQLQESGGGLVQPGGSLRLSCAASGFTFSGVDMSWVRQAPGRGLEWVSGFGSGGESPSYADSVKGRFTISRDNAKKMLYLQMDSLKPEDTAVYYCATRWAEGYWGQGTQVTVSS

SEQ ID NO: 79 - protein, 124 aa, artificial sequence
  9# nanobody
DVQLQESGGGLVQPGGSLRLSCAASGFLFSRYSMSWVRQAPGKGLEWVSGITNGGSITKYADSVKGRFTISRDNAKNTLTLQLNSLKTEDTAMYYCAKGAGEYGPVSDYVFSLRGQGTQVTVSS

SEQ ID NO: 80 - protein, 125 aa, artificial sequence
  10# nanobody
DVQLQESGGGSVQAGGSLRLSCAASGYTIGSNYMGWFRQAPGKEREGVAAIYTGGGNTYYADSVKGRFAISEDNAKNTVYLHMNLLKPEDTAMYYCAADLRLRVAVLHPYGYNYWGQGTQVTVSS

SEQ ID NO: 81 - protein, 113 aa, artificial sequence
  11# nanobody
DVQLQESGGGLVQPGGSLRLSCAVSGFTFSNYAMSWVRQAPGKGLEWVSRISSSGITYYADSVKGRFTISRDNAKNTLYLQLNNLKTEDTAMYYCTKSSEAPGGQGTQVTVSS

SEQ ID NO: 82 - protein, 128 aa, artificial sequence
  12# nanobody
DVQLQESGGGSVQAGGSLRLSCAASGAIYRGNYMGWFRQAPGKEREGVAAIATNGRTTYYADSVKGRFTISQDNAKNTVYLQMNDLKPEDTAMYYCAADNSWSCTVVGGLLAPAYNYWGQGTQVTVSS

SEQ ID NO: 83 - protein, 127 aa, artificial sequence
  13# nanobody
DVQLQESGGGSVQAGGSLRLSCAASRFTYSWYCMGWFRQAPGKEREGVAAICTTPSRTYAADSVKGRFTISLDIAKKTLSLQMDSLKSEDTAMYYCAADTRGAIGFFKGSGPAIPYWGQGTQVTVSS

SEQ ID NO: 84 - DNA, 369 bp, artificial sequence
  DNA of 1# nanobody
gatgtgcagctgcaggagtctggaggaggctcggtgcaggctggagggtctctgagactctcctgtgcagcctctggtgtcgcccacaggcgctactacatgtcctggttccgccaggctccagggaaggagcgcgagggggtcgcatccatttatattggtagtggtagcacagactatgtcgactccgtgaagggccgattcaccatctcccaagacaacgccaacgacatggtgtatctgcaaatgctcagcctcaaacctgaggacactgccatgtactactgtgcggcaggcctttacggtggtagatgggatcgggcccagtataagtactggggccaggggacccaggtcaccgtctcctca

SEQ ID NO: 85 - DNA, 393 bp, artificial sequence
  DNA of 2# nanobody
gatgtgcagctgcaggagtctggaggaggctcggtgcaggctggagggtctctgagactctcctgtacagcctccggatacacccgcagtaccatttggataggctggttccgccaggctccagggaaggagcgcgagggggtcgcagctatttatattggtagtggcgcgacatactatgtggactccgtgaagggccgattcaccatctcccaagacaacgccaagaacgccaagaacacggtgtatctgcagatgaacagcctgaaacctgaggacactgccatgtactactgtgcggcaacgggcggtactgtaggaagtatggtccgattctccccagtgggcgactttggttattggggccaggggacccaggtcaccgtctcctca

SEQ ID NO: 86 - DNA, 369 bp, artificial sequence
  DNA of 3# nanobody
gatgtgcagctgcaggagtctggaggaggcttggtgcagcctggggggtctctgagactctcctgtgcagcctctggattcaccttcacaaactatgccatgagctgggtccgccaggctccagggaagggactcgagtgggtctcaggtattaatagtgatggtggcacatactattcaaactccgtgaagggccgaagcaccatctccagagacaacgccaagaacacgctgtatctgcaattgaacagcctggagactgaggacacggccatgtattactgtgcaaaaggaaacggcccgtcttggaccctggggagcgtggacgttaactcccggggccaggggacccaggtcaccgtctcctca

SEQ ID NO: 87 - DNA, 378 bp, artificial sequence
  DNA of 4# nanobody
gatgtgcagctgcaggagtctggaggaggctcggtgcaggctggagggtctctgagactctcctgtgcagcctctggagacacttacaggcgcaacaccatgggctggttccgccaggctccggggaaggagcgcgagggggtcgcagctatttatactggtggtactagtacagcctatgccgactccgtgaggggccgattcaccatctcccaagacagcgccaagaacacggtgtatctgcaaatgaacagcctgaaacctgaggacactgccatatactactgtgcggcaggctcagtgtggtcccgttatacatggtcctggcccagtacgtatagctactggggccaggggacccaggtcaccgtctcctca

SEQ ID NO: 88 - DNA, 372 bp, artificial sequence
  DNA of 5# nanobody
gatgtgcagctgcaggagtctggaggaggctcggtgcaggctggagggtctctgagactctcctgtgcagcctctgaatacagctacagtaggaagtggatgggctggttccgccaggctccagggaaggagcgcgagggggtcgcagctatttatactagtggtaggacagccgttgccgactccgtgaagggccgattcaccatctcccaagacatcgccgagaacacggtgtatctgcgtatgaacagcctgaaacctgaggacactgccatgtactactgtgcggcactcacaggtgctagcgactcaccgctacgcccaagcgagtataactactggggccaggggacccaggtcaccgtctcctca

SEQ ID NO: 89 - DNA, 390 bp, artificial sequence
  DNA of 6# nanobody
gatgtgcagctgcaggagtctggaggaggctcggtgcaggctggagggtctctgagactctcctgtgcagcctctggattcacattcagtagctacgacatgagctgggtccgccaggctccagggaaggggctcgagtgggtctcagctattaatagtggtggtggtagcacatactatgcagactccgtgaagggccgattcaccatctccagagacaacgccaagaacacgatgtatctgcaaatgaacagcctgaaacctgaggacactgccatgtactactgtgcggcagatcctggttcaagtttggcggtgggagcttggttgaaggcggcggatcccttctatggttactggggccaggggacccaggtcaccgtctcctca

SEQ ID NO: 90 - DNA, 393 bp, artificial sequence
  DNA of 7# nanobody
gatgtgcagctgcaggagtctggaggaggctcggtgcaggctggagggtctctgagactctcctgtacagcctccggatacacccgcagtaccatttggataggctggttccgccaggctccagggaaggagcgcgagggggtcgcagctatttatattggtagtggcgcgacatactatgtggactccgtgaagggccgattcaccatctcccaagacaacgccaagaacgccaagaacacggtgtatctgcagatgaacagcctgaaacctgaggacactgccatgtactactgtgcggcaacgagcggtactgtaggaagtatggtccgattctccccagtgggcgactttggttattggggccaggggacccaggtcaccgtctcctca

SEQ ID NO: 91 - DNA, 345 bp, artificial sequence
  DNA of 8# nanobody
gatgtgcagctgcaggagtctggaggaggcttggtgcagcctggggggtctctgagactctcctgtgcagcctctggattcacattcagtggcgtcgacatgagctgggtccgccaggctccagggagggggctcgagtgggtctcagggtttggtagtggtggtgaaagcccatcatatgcagactccgtgaagggccgattcaccatctccagagacaacgccaagaaaatgctgtatctgcagatggacagtttgaaacctgaggacacagccgtgtattactgcgccacccgctgggctgagggctactggggccaggggacccaggtcaccgtctcctca

SEQ ID NO: 92 - DNA, 372 bp, artificial sequence
  DNA of 9# nanobody
gatgtgcagctgcaggagtctggaggaggcttggtgcagcctggggggtctctgagactctcctgtgcagcctctggattcctcttcagtagatattccatgagttgggtccgccaggctccagggaagggactcgagtgggtctcaggtattacgaatggtggtagcattacaaagtatgcagactccgtgaagggccgattcaccatctccagagacaacgccaagaacacactgactctgcaactgaacagcctgaaaactgaggacacggccatgtattactgtgccaaaggcgcgggggagtatggccctgttagtgactatgtcttctctctcaggggccaggggacccaggtcaccgtctcctca

SEQ ID NO: 93 - DNA, 375 bp, artificial sequence
  DNA of 10# nanobody
gatgtgcagctgcaggagtctggaggaggctcggtgcaggctggagggtctctgagactctcctgtgcagcctctggatacaccatcggtagcaactacatgggctggttccgccaggctccagggaaggagcgcgagggggtcgcagctatttatactggtggtggtaatacatactatgccgactccgtgaagggccgattcgccatctccgaagacaacgccaagaacacggtgtatctgcacatgaacctcctgaaacctgaggacactgccatgtactactgtgcggcagacttgagactacgggttgctgtactccacccgtatgggtataactactggggccaggggacccaggtcaccgtctcctca

SEQ ID NO: 94 - DNA, 339 bp, artificial sequence
  DNA of 11# nanobody
gatgtgcagctgcaggagtctggaggaggcttggtgcagcctggggggtctctgagactctcctgtgcagtctctggattcaccttcagtaactatgccatgagctgggtccgccaggctccagggaaggggctcgagtgggtctcacgtattagtagtagtggtattacatactatgcagactccgtgaagggccgattcaccatctccagagacaacgccaagaacacgctgtatctacaattgaacaacctgaaaactgaggacacggccatgtattactgtacaaaaagttccgaggccccgggtggccaggggacccaggtcaccgtctcctca

SEQ ID NO: 95 - DNA, 384 bp, artificial sequence
  DNA of 12# nanobody
gatgtgcagctgcaggagtctggaggaggctcggtacaggctggagggtctctgagactctcctgtgcagcctctggagccatctacagaggcaactacatgggctggttccgccaggctccagggaaggagcgcgagggggtcgcggctatcgctactaatggtcgtacaacatactatgccgactccgtgaagggccgattcaccatctcccaagacaacgccaagaacacggtgtatctgcaaatgaacgacctgaaacctgaggacactgccatgtactactgtgcggcagacaactcttggtcctgtacggtggtaggtgggctactggcgcctgcctataactactggggccaggggacccaggtcaccgtctcctca

SEQ ID NO: 96 - DNA, 381 bp, artificial sequence
  DNA of 13# nanobody
gatgtgcagctgcaggagtctggaggaggctcggtgcaggctggagggtctctgagactctcctgtgcagcctctagatttacctacagttggtactgcatgggctggttccgccaggctccggggaaggagcgcgagggggtcgcagctatttgcacgacaccgagccgcacatacgctgccgactccgtgaagggtcgattcaccatctccctagacatcgccaagaagacgctgtctctgcaaatggacagcctgaaatctgaggacactgccatgtactactgtgcggcagatacaagaggagctatagggttcttcaagggcagtgggcccgccatcccctactggggccaggggacccaggtcaccgtctcctca

(19) United States
(12) Patent Application Publication
(10) Pub. No.: US 2024/0092910 A1
(43) Pub. Date: Mar. 21, 2024

(54) B7-H3 NANOBODY, PREPARATION METHOD AND USE THEREOF

(71) Applicants: Dartsbio Pharmaceuticals Ltd., Zhongshan City, Guangdong (CN); Shanghai Mabstone Biotechnology Ltd., Shanghai (CN); Shenzhen Innovastone Biopharma Ltd., Shenzhen, Guangdong (CN)

(72) Inventors: Chunhe WANG, Zhongshan City, Guangdong (CN); Yi-li CHEN, Zhongshan City, Guangdong (CN); Xinyuan LIU, Zhongshan City, Guangdong (CN); Weidong LUO, Zhongshan City, Guangdong (CN); Guojian LIU, Zhongshan City, Guangdong (CN); Huanhuan LI, Zhongshan City, Guangdong (CN); Yijun LIN, Zhongshan City, Guangdong (CN)

(73) Assignees: Dartsbio Pharmaceuticals Ltd., Zhongshan City, Guangdong (CN); Shanghai Mabstone Biotechnology Ltd., Shanghai (CN); Shenzhen Innovastone Biopharma Ltd., Futian District, Shenzhen (CN)

(21) Appl. No.: 17/767,444

(22) PCT Filed: Oct. 9, 2020
(86) PCT No.: PCT/CN2020/119893

§ 371 Date: Apr. 8, 2022

(30) Foreign Application Priority Data
Oct. 9, 2019 (CN) 201910953967.4

(51) Int. Cl. C07K 16/28

(52) U.S. Cl. CPC C07K 16/2827; C07K 2317/22; C07K 2317/569; C07K 2317/76

(57) ABSTRACT

The present invention provides a B7-H3 nanobody, the preparation method and use thereof. The B7-H3 nanobody comprises framework regions 1-4 (FR 1-4) and complementarity determining regions 1-3 (CDR 1-3), can specifically bind to B7-H3, and can be used for detecting B7-H3 molecules, and be used for the treatment of various malignant tumors with abnormal expression of B7-H3 molecule.

### TECHNICAL FIELD

The present invention relates to a nanobody against the antigenic epitope of a B7-H3 polypeptide molecule, a preparation method and use thereof.

### BACKGROUND

B7-H3 (also known as CD276), a member of the B7 family, with the sequence similar to the extracellular domain of PD-L1 (B7-H1), is a phylogenetically conserved protein with various biological functions. B7-H3 is a type I transmembrane protein whose extracellular domain in mice consists of a pair of immunoglobulin variable domain (IgV) and immunoglobulin constant domain (IgC) (2IgB7-H3 isoform); in humans, it consists of two pairs of identical immunoglobulins produced by exon duplication (4IgB7-H3 isoform), which isoform is the predominant isoform expressed. B7-H3 is widely expressed at the mRNA level and can be detected in non-lymphoid organs such as liver, heart, prostate, and lymphoid organs such as spleen and thymus, however, its protein expression is strictly regulated. B7-H3 is structurally expressed on non-immune quiescent fibroblasts, endothelial cells, osteoblasts and amniotic fluid stem cells. After induction, B7-H3 can be expressed on immune cells, especially on antigen-presenting cells. Being co-cultured with regulatory T cells (Treg), dendritic cells can be induced to express B7-H3 protein with IFN-γ, lipopolysaccharide (LPS) or anti-CD40 stimulation in vitro. In addition, B7-H3 expression level can be up-regulated after monocytes and monocyte-derived DCs are stimulated by LPS or differentiated as induced by cytokines, respectively; B7-H3 protein can also be detected on NK cells, B cells, and a small number of T cells after PMA/lonomycin stimulation. Unlike restricted regulation in normal cells, B7-H3 is abnormally overexpressed in a variety of malignancies, including melanoma, leukemia, breast, prostate, ovarian, pancreatic, rectal and other cancers. High expression of B7-113 is also associated with poor prognosis and poor clinical efficacy, and the development of antibody drugs targeting B7-H3 has become a promising tumor treatment strategy.

Nanobody technology is an antibody engineering revolution led by biomedical scientists on the basis of traditional antibodies by using molecular biology technology combined with the concept of nanoparticle sciences to develop the latest and smallest antibody molecules. It was originally found in camel blood by Belgian scientist Hamers, R. Ordinary antibody proteins are composed of two heavy chains and two light chains, while the nanoantibodies found in camel blood only have two heavy chains. Like traditional antibodies, these “heavy chain antibodies” can bind tightly to targets such as antigens, meanwhile, unlike single-chain antibodies, they are of a low aggregate tendency. Nanobody based on such “heavy chain antibody” is advantageous not only in a molecular weight that is only 1/10 of that of traditional antibodies, but also in its more flexible chemical properties, good stability, high solubility, easy expression, high tumor tissue penetration and easy coupling to other molecules. Therefore, the application of nanobody technology to develop therapeutic antibodies against B7-H3 has great prospects.

### SUMMARY OF THE INVENTION

In view of the above problems, on the one hand, the present invention provides a B7-H3 nanobody, which comprises framework regions (FR) and complementarity determining regions (CDR), the complementarity determining regions comprise a complementarity determining region 1 (CDR1), a complementarity determining region 2 (CDR2) and complementarity determining region 3 (CDR3), wherein,
the sequence of the complementarity determining region 1 (CDR1) is selected from any one of SEQ ID NO:5, SEQ ID NO:12, SEQ ID NO:19, SEQ ID NO:24, SEQ ID NO:29, SEQ ID NO:34, SEQ ID NO:41, SEQ ID NO:46, SEQ ID NO:51, SEQ ID NO:58, SEQ ID NO:63 and SEQ ID NO:68;
the sequence of the complementarity determining region 2 (CDR2) is selected from any one of SEQ ID NO:6, SEQ ID NO:13, SEQ ID NO:20, SEQ ID NO:25, SEQ ID NO:30, SEQ ID NO:35, SEQ ID NO:42, SEQ ID NO:47, SEQ ID NO:52, SEQ ID NO:59, SEQ ID NO:64, and SEQ ID NO:69; and
the sequence of the complementarity determining region 3 (CDR3) is selected from any one of SEQ ID NO:7, SEQ ID NO:14, SEQ ID NO:21, SEQ ID NO:26, SEQ ID NO:31, SEQ ID NO:36, SEQ ID NO:37, SEQ ID NO:43, SEQ ID NO:48, SEQ ID NO:53, SEQ ID NO:60, SEQ ID NO:65, and SEQ ID NO:70.

In one embodiment, the B7-H3 nanobody comprises the following regions in order: a framework region 1 (FR1), a complementarity determining region 1 (CDR1), a framework region 2 (FR2), a complementarity determining region 2 (CDR2), a framework region 3 (FR3), a complementarity determining region 3 (CDR3) and a framework region 4 (FR4), wherein
the sequence of the framework region 1 (FR1) is selected from any one of SEQ ID NO:1, SEQ ID NO:8, SEQ ID NO:15, SEQ ID NO:22, SEQ ID NO:27, SEQ ID NO:32, SEQ ID NO: 38, SEQ ID NO:44, SEQ ID NO:49, SEQ ID NO:54, SEQ ID NO:61 and SEQ ID NO:66;
the sequence of the complementarity determining region 1 (CDR1) is selected from any one of SEQ ID NO:5, SEQ ID NO:12, SEQ ID NO:19, SEQ ID NO:24, SEQ ID NO:29, SEQ ID NO:34, SEQ ID NO:41, SEQ ID NO:46, SEQ ID NO:51, SEQ ID NO:58, SEQ ID NO:63 and SEQ ID NO:68;
the sequence of the framework region 2 (FR2) is any one selected from any one of SEQ ID NO:2, SEQ ID NO:9, SEQ 10 NO:16, SEQ ID NO:39, and SEQ ID NO:55;
the sequence of the complementarity determining region 2 (CDR2) is selected from any one of SEQ ID NO:6, SEQ ID NO:13, SEQ ID NO:20, SEQ ID NO:25, SEQ ID NO:30, SEQ ID NO:35, SEQ ID NO:42, SEQ ID NO:47, SEQ ID NO:52, SEQ ID NO:59, SEQ ID NO:64 and SEQ ID NO:69;
the sequence of the framework region 3 (FR3) is selected from any one of SEQ ID NO:3, SEQ ID NO:10, SEQ ID NO:17, SEQ ID NO:23, SEQ ID NO:28, SEQ ID NO:33, SEQ ID NO: 40, SEQ ID NO:45, SEQ ID NO:50, SEQ ID NO:56, SEQ ID NO:62 and SEQ ID NO:67;
the sequence of the complementarity determining region 3 (CDR3) is selected from any one of SEQ ID NO:7, SEQ ID NO:14, SEQ ID NO:21, SEQ ID NO:26, SEQ ID NO:31, SEQ ID NO:36, SEQ ID NO:37, SEQ ID NO:43, SEQ ID NO:48, SEQ ID NO:53, SEQ ID NO:60, SEQ ID NO:65, and SEQ ID NO:70; and
the sequence of the framework region 4 (FR4) is selected from any one of SEQ ID NO:4, SEQ ID NO:11, SEQ ID NO:18 and SEQ ID NO:57.

In one embodiment, the B7-H3 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 5,
the sequence of the complementarity determining region 2 (CDR2) is SEQ ID NO: 6,
the sequence of the complementarity determining region 3 (CDR3) is SEQ ID NO:7.

In one embodiment, the B7-H3 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 12,
the sequence of the complementarity determining region 2 (CDR2) is SEQ ID NO: 13,
the sequence of the complementarity determining region 3 (CDR3) is SEQ ID NO:14.

In one embodiment, the B7-13 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 19,
the sequence of the complementarity determining region 2 (CDR2) is SEQ ID NO: 20,
the sequence of the complementarity determining region 3 (CDR3) is SEQ ID NO:21.

In one embodiment, the B7-H3 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 24,
the sequence of the complementarity determining region 2 (CDR2) is SEQ ID NO: 25,
the sequence of the complementarity determining region 3 (CDR3) is SEQ ID NO:26.

In one embodiment, the B7-H3 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 29,
the sequence of the complementarity determining region 2 (CDR2) is SEQ ID NO: 30,
the sequence of the complementarity determining region 3 (CDR3) is SEQ ID NO:31.

In one embodiment, the B7-H3 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 34,
the sequence of the complementarity determining region 2 (CDR2) is SEQ ID NO: 35,
the sequence of the complementarity determining region 3 (CDR3) is SEQ ID NO:36.

In one embodiment, the B7-H3 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 12,
the sequence of the complementarity determining region 2 (CDR2) is SEQ ID NO: 13,
the sequence of the complementarity determining region 3 (CDR3) is SEQ ID NO:37.

In one embodiment, the 67-H3 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 41,
the sequence of the complementarity determining region 2 (CDR2) is SEQ ID NO: 42,
the sequence of the complementarity determining region 3 (CDR3) is SEQ ID NO:43.

In one embodiment, the B7-H3 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 46,
the sequence of the complementarity determining region 2 (CDR2) is SEQ ID NO: 47,
the sequence of the complementarity determining region 3 (CDR3) is SEQ ID NO: 48.

In one embodiment, the B7-H3 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 51,
the sequence of the complementarity determining region 2 (CDR2) is SEQ ID NO: 52,
the sequence of the complementarity determining region 3 (CDR3) is SEQ ID NO: 53.

In one embodiment, the 67-H3 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 58,
the sequence of the complementarity determining region 2 (CDR2) is SEQ ID NO: 59,
the sequence of the complementarity determining region 3 (CDR3) is SEQ ID NO: 60.

In one embodiment, the B7-H3 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 63,
the sequence of the complementarity determining region 2 (CDR2) is SEQ ID NO: 64,
the sequence of the complementarity determining region 3 (CDR3) is SEQ ID NO: 65.

In one embodiment, the B7-H3 nanobody comprises a complementarity determining region 1 (CDR1), a complementarity determining region 2 (CDR2) and a complementarity determining region 3 (CDR3), wherein,
the sequence of the complementarity determining region 1 (CDR1) is SEQ ID NO: 68,
the sequence of the complementarity determining region 2 (CDR2) is SEQ ID NO: 69,
the sequence of the complementarity determining region 3 (CDR3) is SEQ ID NO:70.

In one embodiment, the B7-H3 nanobody has an amino acid sequence selected from any one of the following:
SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82 and SEQ ID NO:83.

In another aspect, the present invention provides a method for preparing the above B7-H3 nanobody, comprising the following steps:
1). Constructing B7-H3 molecular nanobody library;
2). Screening B7-H3 nanobodies;
3). Detecting single positive clones by immunoblotting;
4). Further verifying positive clones with phage Elisa; and
5). Expressing B7-H3 nanobodies in eukaryotic expression system and purifying them.

In another aspect, the present invention provides a DNA molecule encoding the above-mentioned B7-H3 nanobody.

In yet another aspect, the present invention provides an expression vector comprising the above-mentioned DNA molecule.

In yet another aspect, the present invention provides the use of the above-mentioned B7-H3 nanobody in the preparation of a B7-H3 molecule detection reagent.

In another aspect, the present invention provides the use of the above-mentioned B7-H3 nanobody in the preparation of a medicament for the treatment of malignant tumors with high expression level of B7-H3 molecule.

### Technical Effects of the Present Invention

The B7-H3 nanobody of the present invention can specifically bind to B7-H3, therefore, can be used to detect B7-H3 molecules with high sensitivity, and is expected to be used as a therapeutic antibody for the treatment of various malignant tumors with high expression level of B7-H3 molecule.

### DESCRIPTION OF FIGURES

FIG. 1 shows an output phage immunoblot in Example 3.

FIG. 2 shows the results of further verification of positive clones with phage Elisa in Example 4.

FIG. 3 is the SDS-PAGE picture of the purified 1-13 #nanobodies in Example 5.

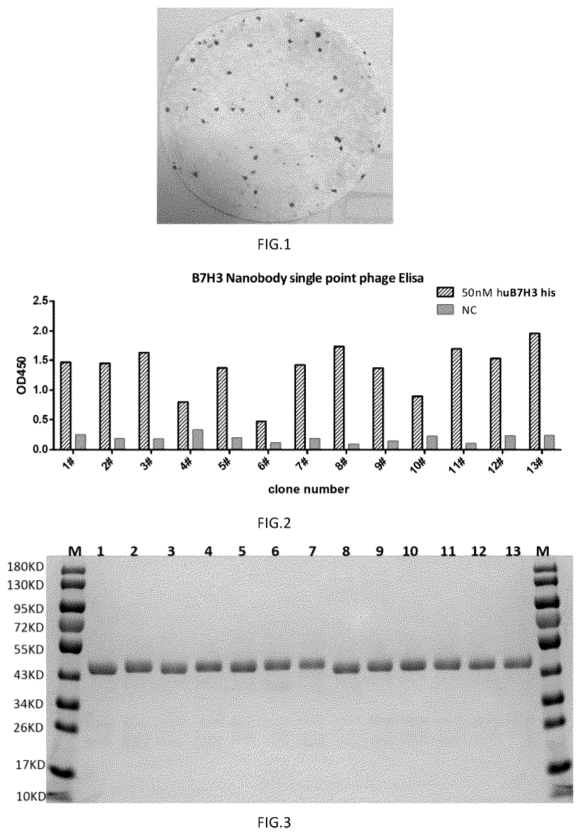

### DETAILED EMBODIMENTS

The specific preparation process of the nanobodies of the present application will be described below with reference to specific examples. However, those skilled in the art should understand that the scope of the present application is not intended to be limited by these examples, but includes various equivalent and modified forms made by those skilled in the art.

Complete/incomplete Freund's adjuvant (sigma, F5506), streptavidin beads (invitrogen, 65002), casein (Thermo, 37528), PROTRAN BA 85 (Whatman, 10401116), microtiter plate (greiner, 650061)), goat anti-M13HRP (GE, 279421).

### Example 1: Construction of Nanobody Library Against B7-H3 Molecular

(1) 1 mg of the hu4IgB7-H3 antigen produced by protein expression was mixed with complete Freund's adjuvant in an equal volume and a Xinjiang dromedary was immunized therewith;
(2) Starting from the second week, the dromedary was immunized for 7 times with a mixture of 1 mg of the hu4IgB7-H3 antigen and incomplete Freund's adjuvant in an equal volume to stimulate B cells to express antigen-specific nanobodies;
(3) After 7 immunizations, 100 ml of the peripheral blood lymphocytes of dromedary was collected for extracting total RNA;
(4) cDNA was obtained by Reverse transcription and amplified by nested PCR to obtain VHH fragments;
(5) VHH single-stranded DNA was annealed into the phage vector using the kunkel reaction;
(6) The kunkel product was transformed into electrotransformation competent DH108, and the B7-H3 nanobody library was constructed and the library size was determined. The library size was 1.07×10e9.

### Example 2: Screening Nanobodies Against B7-H3

(1) Liquid phase panning:
   1. 10e12pfu input phage was blocked with a final concentration of 0.5% casein blocking buffer and incubated for 1 h at room temperature;
   2. At the same time, 100 μl streptavidin magnetic beads were washed three times with sterile PBS, the supernatant was discarded, and the resultant was blocked with 500 μl of 1% casein blocking buffer for 1 hour at room temperature;
   3. The blocking solution in step 2 was discarded, 1 ml of the blocked phage was added to the magnetic beads, mixed well, rotated and mixed for 15 minutes to allow binding; (negative screening)
   4. The supernatant was added into a new sterile EP tube, hu4IgB7-H3-biotin antigen with a final concentration of 100 nM was added thereto, and the resultant was rotated and mixed at room temperature for 2 h to perform binding;
   5. At the same time, 100 μl streptavidin magnetic beads were washed three times with sterile PBS, the supernatant was discarded, and the resultant was blocked with 5001p of 1% casein blocking buffer for 1 h at room temperature;
   6. The blocking solution in step 5 was discarded, 1 ml of the phage-hu hu4IgB7-H3-biotin mixture was added to the magnetic beads, mixed well, rotated and mixed for 15 minutes to perform binding;
   7. The supernatant was discarded and the magnetic beads were washed 10 times with 1×PBS, and collected;
   8. 400 μl of freshly prepared elution buffer containing 100 mM Triethylamine was added to the magnetic beads obtained in step 7, rotated and eluted for 10 min;
   9. 400 μl of the supernatant was transferred to a sterile EP tube, 200 μl of pH6.4 Tris-HCl was added for neutralization, after the screening, the output phage was obtained.
(2) Solid phase panning:
   1. An immunotube was coated with 1 ml of 100 nM hu-hu4IgB7-H3 antigen solution, and incubated at 4° C. overnight;
   2. The next day, the uncoated immune tube was rinsed with 1×PBS for 3 times, spun dry, and blocked with 4 ml of 1% casein blocking buffer with rotation at room temperature for 1 h;
   3. 5×10e12 pfu first-round input phage was blocked with a final concentration of 1% BSA, supplemented with 1×PBS to a final volume of 1 ml, and rotated at room temperature for 1 h for blocking;
   4. The blocking solution in the antigen-uncoated immunotube was discarded, the blocked phage was put into an antigen-uncoated immunotube, rotated for 2 h for binding (negative screening);
   5. At the same time, the coating solution in the antigen-coated immunotube was discarded, and the resultant was rinsed three times with 1×PBS, spun dry, and blocked with 4 ml of blocking buffer with rotation at room temperature for 1 hour;
   6. The blocking solution in the antigen-coated immunotube was discarded, and the negatively screened phage was transferred to the immunotube, and rotated for 2 h for binding;
   7. After discarding the supernatant, the immunotube was rinsed 10 times with 1×PBST, and spun dry;
   8. 1 ml of freshly prepared elution buffer containing 100 mM Triethylamine was added to the immunotube and incubated at room temperature for 10 min;
   9. 1 ml of eluate was transferred to a sterile EP tube, and neutralized with 500 μl pH6.4 Tris-HCl. After the screening, output phage was obtained.

### Example 3: Detection of Single Positive Clones by Immunoblotting (Mccafferty J, Griffiths A D, Winter G, et al. Phage Antibodies: Filamentous Phage Displaying Antibody Variable Domains[J]. Nature, 1990, 348(6301):552-554. Huse W, Sastry L, Iverson S, et al. Generation of a Large Combinatorial Library of the Immunoglobulin Repertoire in Phage Lambda[J]. Biotechnology, 1989, 246(4935):1275-1281.)

(1) 2000pfu output phage was plated on each LB plate and incubated at 37° C. for 6-8 h;
(2) Whatman 0.45 μm NC membrane was coated with 5 ml of 2 μg/ml anti-M13 antibody and incubated at room temperature for 25 h;
(3) The coating solution was discarded, the NC membrane was blocked with 5 ml of 1% casein blocking buffer, and incubated at room temperature for 1 h;
(4) The blocking solution was discarded, and the NC membrane was washed three times with 1×PBS, and dried in air;
(5) The NC membrane was attached on the LB plate grown with phage, punched holes for positioning, and incubated at room temperature overnight;
(6) The next day, the membrane was removed and placed in a new petri dish, washed 3 times with 1×PBS, and incubated in 100 nM hu4IgB7-H3-biotin for 1 h at room temperature;
(7) The membrane was washed 8 times with 0.1% PBST, added with 1:1000 diluted Neutravidin-AP and incubated at room temperature for 30 min;
(8) The membrane was washed 8 times with 0.1% PBST, added with 10 ml AP chromogenic substrate (100 μl BCIP+100 μl NBT), and developed color at room temperature for 10-30 min;
(9) The positive clones were picked out for plaque PCR and sequencing;
(10) The Sequences were aligned using DNAman sequence alignment software, and clones with the same CDR1, CDR2, and CDR3 were considered as the same clone.

### Example 4: Further Verification of Positive Clones with Phage ELISA

(1) Amplification of phage: XL1blue E. coli was seeded at 1:100 in LB liquid medium (containing 10 μg/ml tetracycline), grown to OD60=0.6; XI1blue were transferred into 96-well deep-well plates, 600 μl/well, added with 15 μl of the picked phage elution for amplification overnight. The resultant was centrifuged at 4000 rpm for 30 min, and the supernatant was used for phage Elisa;
(2) Elisa detection:
   1. The hu4IgB7-H3 antigen was diluted with 1×PBS to 100 nM, the ELISA plate was coated with 50 μl of the diluted solution per well, and incubated overnight at 4′C;
   2. After discarding the coating solution, the ELISA plate was spun dry, and rinsed 3 times with 1×PBS;
   3. 200 μl of 1% casein blocking buffer was added to each well for blocking at room temperature for 1 hour;
   4. After discarding the blocking solution, each well was added with 50 μl of high-titer phage, and incubated at room temperature for 2 hours;
   5. The ELISA plate was rinsed 6 times with 0.1% PBST and spun dry;
   6. 1:5000 diluted goat anti-M13-HRP antibody was added at 50 μl/well, and incubated at room temperature for 1 h. The plate was washed 6 times with 0.1% PBST and spun dry;
   7. 50 μl of TMB color developing solution was added to each well for developing color at room temperature for 5 minutes. 50 μl of 2M H₂SO₄ was added to stop the reaction, and the OD450 value was read;
   8. The sample wells having OD values 3 times greater than the control well were regarded as positive clones, and 13 positive clones were finally obtained. (The DNA sequences of the 13 nanobodies obtained are SEQ ID No.: 84-96, respectively.)

### Example 5: Expression of the Nanobodies in Eukaryotic Expression System and Purification Thereof

(1) The VHH fragments of different clones obtained by the previous sequencing analysis were cloned into the PINfuse eukaryotic expression vector;
(2) After sequencing and verifying, the plasmid was extracted;
(3) HEK293F suspension cells were cultured in Freestyle 293 expression medium, when the density reached 1×10e6/ml, and the survival rate was >90%, they were ready for transfection;
(4) The mass ratio of PEI:plasmid was 3:1, and transfection was carried out at the ratio of 1 μg plasmid/1 ml HEK293F cells;
(5) On the 5th-6th day after transfection, cell supernatant was collected, purified with proteinA strain to obtain high-purity antibody protein, and the elution buffer was replaced with PBS using an ultrafiltration column. (The amino acid sequences of the 13 nanobodies obtained are SEQ ID No.: 71-83, respectively.)

1. A B7-H3 nanobody, which comprises framework regions (FR) and complementarity determining regions (CDR), the complementarity determining regions comprise a complementarity determining region 1 (CDR1), a complementarity determining region 2 (CDR2) and complementarity determining region 3 (CDR3), wherein,
the sequence of the complementarity determining region 1 (CDR1) is SEQ ID NO: 5,
the sequence of the complementarity determining region 2 (CDR2) is SEQ ID NO: 6,
the sequence of the complementarity determining region 3 (CDR3) is SEQ ID NO:7;
or
the sequence of the complementarity determining region 1 (CDR1) is SEQ ID NO: 12,
the sequence of the complementarity determining region 2 (CDR2) is SEQ ID NO: 13,
the sequence of the complementarity determining region 3 (CDR3) is SEQ ID NO:14;
or
the sequence of the complementarity determining region 1 (CDR1) is SEQ ID NO: 19,
the sequence of the complementarity determining region 2 (CDR2) is SEQ ID NO: 20,
the sequence of the complementarity determining region 3 (CDR3) is SEQ ID NO:21;
or
the sequence of the complementarity determining region 1 (CDR1) is SEQ ID NO: 24,
the sequence of the complementarity determining region 2 (CDR2) is SEQ ID NO: 25,
the sequence of the complementarity determining region 3 (CDR3) is SEQ ID NO:26;
or
the sequence of the complementarity determining region 1 (CDR1) is SEQ ID NO: 29,
the sequence of the complementarity determining region 2 (CDR2) is SEQ ID NO: 30,
the sequence of the complementarity determining region 3 (CDR3) is SEQ ID NO:31;
or
the sequence of the complementarity determining region 1 (CDR1) is SEQ ID NO: 34,
the sequence of the complementarity determining region 2 (CDR2) is SEQ ID NO: 35,
the sequence of the complementarity determining region 3 (CDR3) is SEQ ID NO:36;
or
the sequence of the complementarity determining region 1 (CDR1) is SEQ ID NO: 12,
the sequence of the complementarity determining region 2 (CDR2) is SEQ ID NO: 13,
the sequence of the complementarity determining region 3 (CDR3) is SEQ ID NO:37;
or
the sequence of the complementarity determining region 1 (CDR1) is SEQ ID NO: 41,
the sequence of the complementarity determining region 2 (CDR2) is SEQ ID NO: 42,
the sequence of the complementarity determining region 3 (CDR3) is SEQ ID NO:43;
or
the sequence of the complementarity determining region 1 (CDR1) is SEQ ID NO: 46,
the sequence of the complementarity determining region 2 (CDR2) is SEQ ID NO: 47,
the sequence of the complementarity determining region 3 (CDR3) is SEQ ID NO: 48;
or
the sequence of the complementarity determining region 1 (CDR1) is SEQ ID NO: 51,
the sequence of the complementarity determining region 2 (CDR2) is SEQ ID NO: 52,
the sequence of the complementarity determining region 3 (CDR3) is SEQ ID NO: 53;
or
the sequence of the complementarity determining region 1 (CDR1) is SEQ ID NO: 58,
the sequence of the complementarity determining region 2 (CDR2) is SEQ ID NO: 59,
the sequence of the complementarity determining region 3 (CDR3) is SEQ ID NO: 60;
or
the sequence of the complementarity determining region 1 (CDR1) is SEQ ID NO: 63,
the sequence of the complementarity determining region 2 (CDR2) is SEQ ID NO: 64,
the sequence of the complementarity determining region 3 (CDR3) is SEQ ID NO: 65;
or
the sequence of the complementarity determining region 1 (CDR1) is SEQ ID NO: 68,
the sequence of the complementarity determining region 2 (CDR2) is SEQ ID NO: 69,
the sequence of the complementarity determining region 3 (CDR3) is SEQ ID NO:70.

2-15. (canceled)

16. The B7-H3 nanobody according to claim 1, wherein, the B7-H3 nanobody has an amino acid sequence selected from any one of the following:
SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82 and SEQ ID NO:83.

17. (canceled)

18. A DNA molecule encoding the B7-H3 nanobody of claim 1.

19. An expression vector comprising the DNA molecule of claim 18.

20. A method of preparing a B7-H3 molecule detection reagent using the B7-H3 nanobody of claim 1.

21. (canceled)

22. A DNA molecule encoding the B7-H3 nanobody of claim 16.

23. A method of preparing a B7-H3 molecule detection reagent using the B7-H3 nanobody of claim 16.
